(19) United States
(12) Patent Application Publication
(10) Pub. No.: US 2005/0287066 A1
(43) Pub. Date: Dec. 29, 2005

(54) Anti-rat angiotensin I-converting enzyme (ACE, CD143) monoclonal antibodies and uses thereof

(75) Inventors: Sergei Danilov, Oak Park, IL (US); Irina V. Balyasnikova, Chicago, IL (US)

Correspondence Address: Benjamin Aaron Adler;ADLER & ASSOCIATES, 8011 Candle Lane, Houston, TX, 77071, US

(73) Assignee: MonoACE, Inc.

(21) Appl. No.: 11/169,108

(22) Filed: Jun. 28, 2005

Related U.S. Application Data
(60) Provisional application No. 60/583,337, filed on Jun. 28, 2004.

(51) Int. Cl. A61K 51/00; G01N 33/53; G01N 33/567; A61K 39/395

(52) U.S. Cl. 424/1.49; 424/146.1; 435/7.2; 530/388.26

(57) ABSTRACT

It was demonstrated previously that mAb 9B9 to ACE accumulated selectively in the rat lung after systemic injection and thus could be used as a powerful tool for immunotargeting therapeutic agents/genes to the lung microvasculature. The current invention generated novel set of monoclonal antibodies (mAbs) to rat ACE in order to enhance the repertoire of mAbs suitable for targeting drugs/genes to the rat lung. Provided are methods used to characterize the ability of these mAbs to target ACE in vitro and to the lung endothelium in vivo. The anti-rat ACE mAbs of the present invention demonstrated highly efficient and selective accumulation in the lung.

### CROSS-REFERENCE TO RELATED APPLICATION

This non-provisional application claims benefit of provisional patent applications U.S. Ser. No. 60/583,337, filed Jun. 28, 2004, now abandoned.

### BACKGROUND OF THE INVENTION

1. Field of the Invention

The present invention relates generally to the field of immunology. More specifically, the present invention generates five new monoclonal antibodies (mAbs) directed against different epitopes on rat angiotensin-converting enzyme and demonstrates their utility in imaging of lung vessels and targeting drugs/genes to the pulmonary vasculature.

2. Description of the Related Art

Angiotensin-converting (ACE) enzyme is an important regulator of blood pressure (Soffer R. L., 1991; Edberg J. C. et al., 1989; Corvol P. et al., 1995). Besides its abundant epithelial expression, this transmembrane glycoprotein is also expressed on the luminal surface of endothelial cells (Caldwell P. R. et al., 1976; Ryan U. S. et al., 1976). The pulmonary vasculature is enriched in ACE: nearly 100% of the endothelial cells in the alveolar capillaries are ACE-positive, in contrast to 10-15% in the extra-pulmonary capillaries (Franke F. E. et al., 1997, Danilov S. M. et al., 1989). Thus, endothelial ACE is expressed rather heterogeneously in a vessel- and species-specific manner (Morrel N. W. et al., 1995; Franke F. E. et al., 1997; Danilov S. M. et al., 1989).

Various studies have demonstrated ACE to be a suitable target for the specific delivery of drugs to the lung vasculature using anti-ACE mAbs as carriers (Danilov S. M. et al., 1988; Danilov S. et al., 1989; Muzykantov V. et al., 1994; Muzykantov V. R. and Danilov S. M., 1995). Systemic injection of anti-ACE mAb 9B9 led to selective accumulation of the mAb in the lungs of several mammals (Danilov et al., 1991; Danilov S. et al., 1994) including humans (Muzykantov and Danilov, 1995). Some studies used radiolabeled anti-ACE mAbs and reporter compounds conjugated with anti-ACE mAbs to demonstrate accumulation of the mAb in the lungs of animals and humans (Danilov et al., 1989; Danilov et al., 1994, Muzykantov et al., 1996; Muzykantov et al., 1996). Additionally, it was also observed that anti-ACE mAbs did not cause acute harmful reactions in animals or humans (Danilov S. et al., 1994; Muzykantov and Danilov, 1995). Further, since endothelial cells internalized anti-ACE mAbs, it was contemplated that these mAbs could be used to deliver drugs intracellularly (Muzykantov et al., 1996).

Conjugation of anti-ACE mAb 9B9 to plasminogen activators, catalase or superoxide dismutase demonstrated specific targeting of these drugs and prolonged association with the pulmonary vasculature (Muzykantov V. et al., 1996, Muzykantov V. R. et al., 1996). The therapeutic relevance of this approach was supported by the observation that conjugates of catalase with anti-ACE mAb 9B9 accumulated in rat lungs in vivo and diminished the damage of the endothelium by hydrogen peroxide in isolated perfused lung (Atochina E. N. et al., 1998).

Anti-ACE antibodies were successfully used to redirect viral vectors to the rat lung and increase the selectivity and efficacy of transgene expression that had been incorporated into viral vectors (Reynolds P. N. et al., 2001, Reynolds P. N. et al., 2000). Thus, using a bi-specific conjugate for re-direction of adenoviruses to ACE, a 20-fold enhancement of pulmonary gene delivery and expression in vivo, along with significantly reduced (5-8-fold) transgene expression in non-targeted organs was achieved (Reynolds P. N. et al., 2001). Moreover, the combination of transductional retargeting adenoviruses (via ACE) and transcriptional retargeting (with the use of an endothelial specific promoter for vascular endothelial growth factor receptor1, flt-1) resulted in a remarkable, highly synergistic improvement in selectivity of transgene expression in the lung compared to the usual site of vector sequestration, the liver. This was demonstrated by improvement in relative selectivity of 300,000-fold for lung:liver expression and 6000-fold for lung:spleen expression when compared to non-targeted vector (Reynold P. N. et al., 2001). Thus, antibody-directed lung-selective gene delivery via ACE showed tremendous potential (Pinckard R. N. and Weir D. M., 1978; Pimm M. V., 1995). As a confirmation of this potential, it was observed that systemic administration of adenoviruses encoding eNOS, chemically conjugated with anti-ACE mAb 9B9, enhanced eNOS expression in the rat lung and attenuated the systemic hypertension in SHR-SP rats.

To date, anti-ACE mAb 9B9 is the only mAb known to react with rat ACE and accumulate in the rat lung after systemic injection (Danilov et al., 1991; Danilov et al., 1994). Therefore, there was a necessity to enhance the repertoire of mAbs that detect different epitopes of rat ACE, which are highly specific and suitable for targeting drugs/genes to the rat lung. Additionally, it was also demonstrated that the binding of mAbs to different epitopes of human ACE resulted in different, sometimes opposite effects on ACE dimerization, shedding and activity (Balyasnikova et al., 2002; Kost et al., 2003). It is also known that affinity carriers that induce diverse functional effects on target molecules enhance the therapeutic potential of vascular immunotargeting strategies by helping define the optimal carriers for producing beneficial effects.

The prior art is deficient in monoclonal antibodies that recognize different epitopes of rat ACE. The present invention fulfills this long-standing need and desire in the art by generating new mAbs directed against different epitopes on rat ACE in vitro and characterizing their ability to target ACE in vitro and to the lung endothelium in vivo.

### SUMMARY OF THE INVENTION

The present invention is directed to the generation of novel set of mAbs to rat ACE in order to enhance the repertoire of mAbs suitable for targeting drugs/genes to the lungs. The new mAbs that were generated recognized different epitopes on rat ACE and were examined for their efficacy to bind rat ACE in vitro and in vivo. The present invention demonstrated that gene delivery into cultured rat lung endothelial cells increased 30-50 fold after coating modified adenoviruses (containing Ig-binding domain) with mAbs to rat ACE. Radiolabeled mAbs specifically accumulated in the lung after systemic injection. MAbs 1A2, 4H3 and 2E1 of the present invention demonstrated the highest efficacy of lung uptake-around 50% of injected dose per gram of tissue; for mAb 1A2, the selectivity of lung uptake (ratio of lung to blood radioactivity) was 205. The effect of mAbs on ACE shedding was epitope-specific: injection of mAb 1A2 and 4H3 did not change lung ACE, whereas injection of mAb 2E1 and 9B9 decreased rat lung ACE activity by 20%. However, none of the tested mAbs inhibited ACE activity in vitro.

In one embodiment of the present invention, there is a monoclonal antibody that binds a specific epitope in the N-domain of ACE. In another embodiment of the present invention, there is a method of isolating lung endothelial cells in a sample. This method comprises incubating the sample with anti-ACE monoclonal antibody that binds a specific epitope in the N-domain of ACE. The endothelial cells in the sample are further sedimented using magnetic beads coated with anti-mouse IgG. The endothelial cells that are positively labeled with the magnetic beads linked to the anti-ACE monoclonal antibody are then separated, thereby isolating the lung endothelial cells in the sample.

In still another embodiment of the present invention, there is a method of diagnostic imaging of lung vessel. This method comprises administering radiolabeled anti-ACE monoclonal antibody where the radiolabeled anti-ACE monoclonal antibody binds a specific epitope in the N-domain of ACE on the endothelial cells in lung. The detection of the bound radiolabeled anti-ACE monoclonal antibody enables the imaging of lung vessels.

### BRIEF DESCRIPTION OF THE DRAWINGS

FIGS. 1A-C show binding of mAbs to ACE. FIG. 1A shows results of ELISA where MAbs (10 μg/ml) were incubated in microtiter plates covered by rat lung ACE. The mAb binding was quantified using anti-mouse IgG conjugated with alkaline phosphatase. The binding of control mouse IgG was subtracted from that of mAb binding. FIG. 1B shows results of plate precipitation assay (PPA) where rat lung homogenates were incubated in wells covered by different mAbs to rat ACE (5 μg/ml) via rabbit anti-mouse antibodies. Precipitated ACE activity was further estimated using enzymatic assay. FIG. 1C compares the serum/lung ratio obtained by performing plate precipitation assay. ACE activity precipitated from rat serum and rat lung homogenates by mAbs was estimated and the ratio was used to define the binding characteristics of mAbs to soluble and membrane-bound rat ACE in solution.

FIG. 2 shows antigenic regions of rat ACE. Rat ACE was pre-incubated with 100-fold excess of the indicated protecting antibodies and then added to microtiter wells coated with the indicated test antibodies. Binding of rat ACE to the wells was quantified and bars indicate the percentage of ACE precipitation in the presence of protecting antibody; these values varied by <10% between experiments. 100%-precipitation of rat ACE by the test mAb in the absence of any protecting antibody. Black bars represent precipitation of rat ACE by the test mAb in the presence of an excess of the same protecting mAb. The differences between the triplicates in each of the three separate experiments did not exceed 5-7%.

FIG. 3 is a diagram showing the putative spatial relationship between epitopes. This schematic representation of mAb binding was based on combined results of species and epitope specificity studies (table 1, FIGS. 1 and 2). Overlapping circles represent competitive binding whereas non-overlapping circles indicate non-competitive binding. The adjacent mAbs that recognize identical antigenic determinants is shown (=).

FIGS. 4A-D show binding of mAbs to rat lung endothelial cells. FIG. 4A shows results of Cell ELISA where mAbs (0.1 to 10 μg/ml) were incubated at 4° C. for 1 hour with cultured rat lung microvascular endothelial cells (RLMEC) grown on 96-well microtiter plates. The cells used (6^{th} passage) had a membrane bound ACE activity equal to 23mU/mg. The binding of mAbs was quantified using anti-mouse IgG conjugated with alkaline phosphatase. Bars show mAbs binding at 10 μg/ml. Kₐₚₚ was calculated as previously described (Pinckard R. N and Weir D. M., 1978). FIG. 4B shows results of magnetic beads assay. Capillaries isolated from rat lungs and digested with collagenase were incubated with different mAbs (10 μg/ml) on ice for 1 hour. Magnetic beads coated with anti-mouse IgG were then added. ACE-positive cells covered with mAb-coated beads were separated from contaminating cells using a magnet. Selected endothelial cells were lysed with CHAPS and ACE activity was measured using enzymatic assay. FIG. 4C shows results of mAb-induced ACE shedding. Cultured RLMEC (6^{th} passage) were incubated with mAbs (10 μg/ml) in serum-free medium for 6 hours. Supernatants were collected and ACE activity released from the cell surface into the culture medium was quantified enzymatically by fluorimetric assay. The data in FIGS. 4A, 4B, 4C are mean±SD of triplicates. FIG. 4D shows results of in vitro gene transfer. Rat lung microvacular endothelial cells were infected with modified adenoviruses, AD5DRLLCd, containing an IgG-binding domain on the surface, which were pre-incubated with different mAbs to rat ACE or with diluent. Luciferase assay was performed 48 hours later. Data is presented as a ratio of luciferase expression after coating of Ad vector by mAbs to ACE over that of untreated viruses.

FIGS. 5A-F show immunoreactivity of ACE mAbs in the rat lung. Serial sections of rat lung incubated with new anti-rat ACE mAbs 1A2, 4H3, 5B11, 2E1 shown in FIGS. 5A-5D was compared to the mAb 9B9 shown in FIG. 5E and a negative control MR12/53 shown in FIG. 5F. Endothelial cells of lung capillaries were homogenously and strongly labeled by all mAbs to ACE. Staining of subepithelial capillaries within the microcirculation is shown by arrow. Original magnification ×200, APAAP

FIGS. 6A-C show results of targeting of anti-rat ACE mAbs to lung. I¹²⁵-labeled mAbs (0.5 μCi) were injected i.v. and tissue distribution of radioactivity was estimated 2 hours later. FIG. 6A compares efficacy of lung accumulation, which was expressed as a % of injected dose per gram of tissue (% ID/g). FIG. 6B compares specificity of lung accumulation. The ratio of lung uptake of I¹²⁵-labeled mAbs to that in blood (localization ratio) was calculated in order to determine the antibody with a best specificity of accumulation on the lung. FIG. 6C compares radioresistance of mAbs. Native and radiolabeled mAbs were incubated in rat ACE covered microtiter plates. The binding of native mAbs was quantified using anti-mouse IgG conjugated with alkaline phosphatase. The bound radiolabeled mAbs were eluted with hot 10% SDS and activity of samples was counted by gamma counter. Ratio of radiolabeled mAb binding to rat ACE adsorbed on plastic (RIA) to that of native mAbs (ELISA) was determined as a parameter reflecting a sensitivity of mAbs to denaturation by radioiodination (Balyasnikova I. V. et al., 1999). The data presented are mean±SD of triplicates.

FIG. 7 shows biodistribution of I¹²⁵-labeled mAb 1A2 in rat tissues. I¹²⁵-labeled mAb 1A2 (0.5 μCi) was injected into the tail vein of rats. Two hours later, animals were sacrificed and radioactivity in the specified organs was counted by gamma counter. Tissue uptake was expressed as a % of the injected dose per gram of tissue (% ID/g). The data presented is mean±SD of triplicates.

FIGS. 8A-D show effect of injecting mAbs on rat ACE. MAbs were injected (3 mg/rat) i.v. via the tail vein. The animals were sacrificed four hours later and plasma and organs were removed. Tissue homogenates were prepared in 50mM Tris-HCl buffer (pH 7.3) containing 0.5% Triton X-100 and ACE activity was measured enzymatically by fluorimetric assay. The ACE activity measured in the lung homogenates are shown in FIG. 8A, in the plasma homogenates in FIG. 8B, in the spleen homogenates in FIG. 8C and in the liver in FIG. 8D.

### DETAILED DESCRIPTION OF THE INVENTION

ACE represents one of the most suitable antigens for immunological targeting to the lung vasculature using monoclonal antibodies. However, to date only one mAb directed to human ACE (9B9), which also cross-reacts with rat ACE had been used to investigate its fate and to explore its ability to accumulate efficiently and selectively in the rat lung after systemic injection (Danilov S. et al., 1994; Danilov S. et al., 1989; Danilov S. M. et al., 1991). Therefore, the goal of the present invention was to increase the repertoire of highly specific and efficient mAbs targeting rat ACE in vivo and to investigate the potential of epitope-dependent targeting of different anti-ACE mAbs.

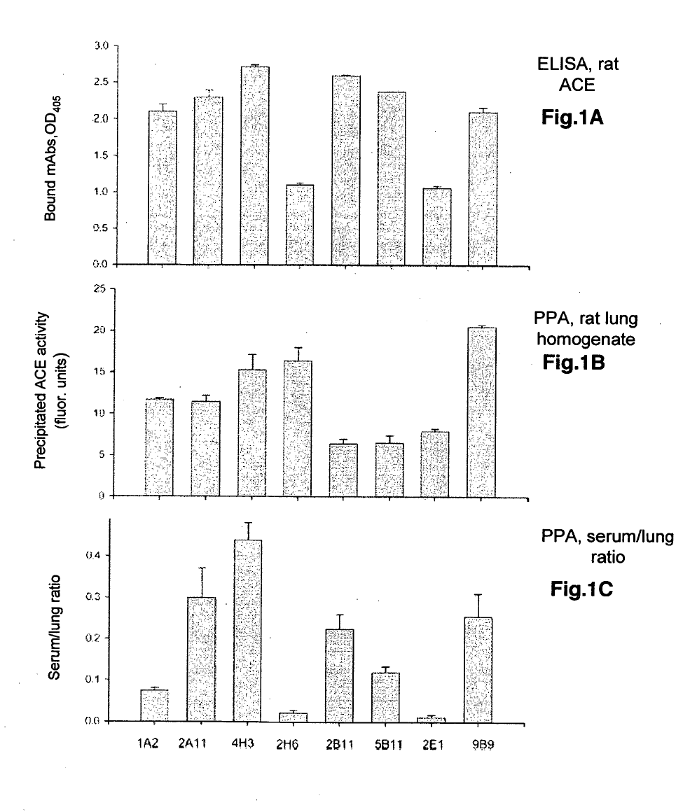

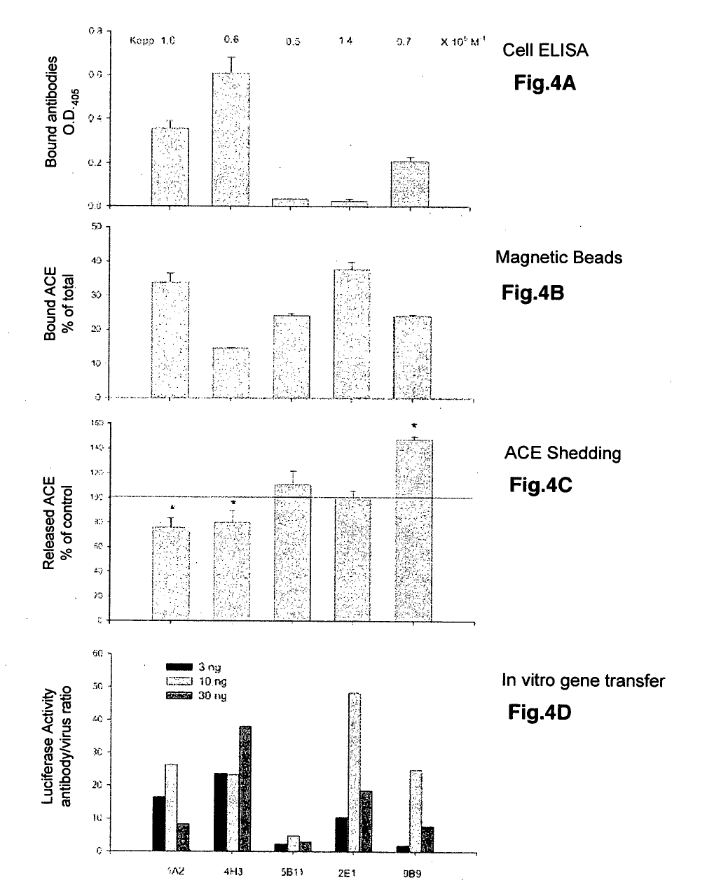

Five new mAbs that recognized different epitopes of rat ACE were generated in the present invention. These mAbs were developed against native rat ACE and selected in vitro using rat ACE in ELISA and plate precipitation assay. All five mAbs were further characterized in terms of endothelial cell surface binding in vitro using cell ELISA and targeting to lung in rats in vivo. Although the present invention intended to choose the best mAb capable of targeting to lungs of rats, in vitro protocols that could predict the mAb that would be most effective and selective in targeting to lungs in vivo was also developed. It was observed that binding of the mAbs depended greatly on the form of ACE that was used for the assays (FIG. 1). Further, in order to determine the suitability of the mAbs for lung endothelial targeting, additional screening assays using rat lung endothelial cells that naturally express ACE on the cell surface were performed (FIG. 4). However, one of the obstacles in mAb-driven delivery to the cell surface was a phenomenon of antigenic modulation. It was previously shown that injection of mAb 9B9 into rat circulation induced shedding of ACE from the surface of lung endothelial cells (Danilov S. et al., 1994). In contrast to this, mAb 1A2 and 4H3 decreased ACE release by 27% as was previously described for anti-human mAb 3G8 (Balyasnikova I. V. et al., 2002; Kost O. A. et al., 2003), thereby indicating that targeting of these mAbs to lungs should not be compromised by induction of ACE shedding upon binding.

Different effects of mAbs (to different epitopes of ACE) on cell-bound ACE activity might be explained either by differential effects on ACE shedding, as was demonstrated previously (Balyasnikova et al., 2002) or by differing abilities to induce internalization of ACE (as a complex with mAbs or separately). However, although the ability to induce internalization differs dramatically between antigens (Danilov et al., 2001), it differs much less dramatically between different mAbs to the same antigen. Additionally, it was also demonstrated previously that the degree of internalization of 8 different mAbs to human ACE from the surface of ACE expressing cells was similar (Balyasnikova et al., 1999). Furthermore, when the internalization of mAbs to rat ACE was estimated, there were no dramatic differences between the 5 mAbs; the degree of internalization was around 30%. Therefore, the results indicated that the differential effects of mAbs to the different epitopes of rat ACE on the membrane-bound ACE activity was explained primarily by their differential effects on ACE shedding rather than on internalization of rat ACE from the cell surface.

Further, it is known that although endothelial cells obtained from various blood vessels express the active enzyme in culture (Johnson A. R. et al., 1980; Del Veccio P. J. and Smith J. R., 1982), they rapidly lose this ability in vitro (Chesterman C. N. et al., 1983; Rosen E. M. et al., 1985; Balyasnikova I. V. et al., 1998). Therefore, the ability of the mAbs of the present invention to isolate rat lung endothelial cells that retained a significant level of ACE expression was also examined. mAbs 1A2 and 2E1 showed the highest ability to enrich a population of ACE-positive rat lung endothelial cells.

The primary application of lung endothelial targeting via ACE is selective delivery of “therapeutic genes” into the lung vasculature (Reynolds P. N. et al., 2000, Reynolds P. N. et al., 2001). However, the efficacy of gene delivery using immunotargeting depends not only on high binding of particular mAbs to the cell surface, but also on the internalization efficacy of the mAb. Therefore, in order to compare the efficacy of gene transfer into rat endothelial cells with mAbs that recognized different epitopes on ACE, a modified adenovirus that expressed immunoglobulin (Ig)-binding domain on the viral surface (Ohno K. et al., 1997; Reynolds P. N. et al., 2001; Soffer R. L., 1976; Khorokhov N. et al., 2003) was used. It was observed that the modified adenovirus expressing Ig-binding C-domain of protein A (Khorokov N. et al., 2003) increased the infectivity of rat lung microvascular endothelial cells upto 50-fold after coating with mAbs to rat ACE (FIG. 4D). Additionally, it was also observed that four mAbs (1A2, 4H3, 2E1 and 9B9) demonstrated comparable efficacy in facilitating gene transfer.

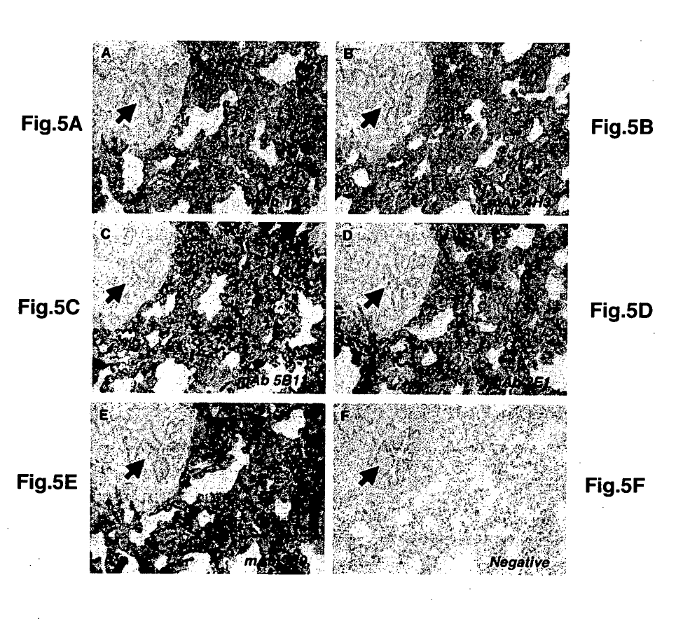

Further, to better characterize the mAbs suitable for lung targeting, the binding of the anti-rat ACE mAbs in rat lung tissue was examined. The results of immunostaining (FIG. 5) with mAbs to rat ACE confirmed the previous finding with mAb 9B9 (Danilov S. M. et al., 2001) that (1) in contrast to the human endothelium (Franke F. E. et al., 1997), the rat endothelium displayed a stronger and more homogenous ACE expression in large vessels, (2) ACE expression in rat capillaries was similar to that seen in human tissues (10% ACE-positive capillaries in extrapulmonary tissues and 100%-positive in lung alveolar capillaries; hepatic and renal capillaries were ACE negative) (Danilov S. M. et al., 2001). This heterogenous endothelial ACE distribution in different organs could provide explanation for increased gene transfer into lung endothelial cells and decreased viral accumulation and transgene expression in the liver that was observed after systemic injection of adenoviruses coated with mAb to ACE (9B9) (Reynolds P. N. et al., 2000; Reynolds P. N. et al., 2001).

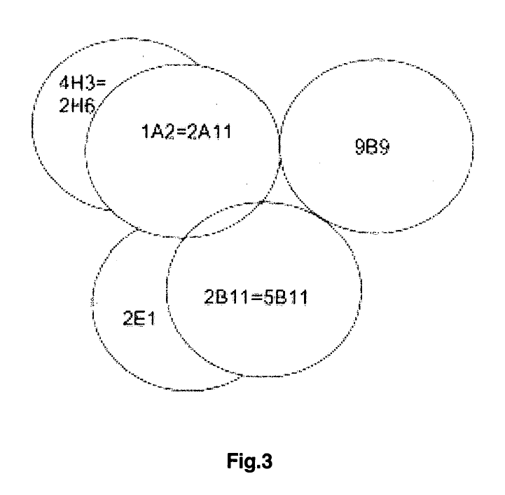

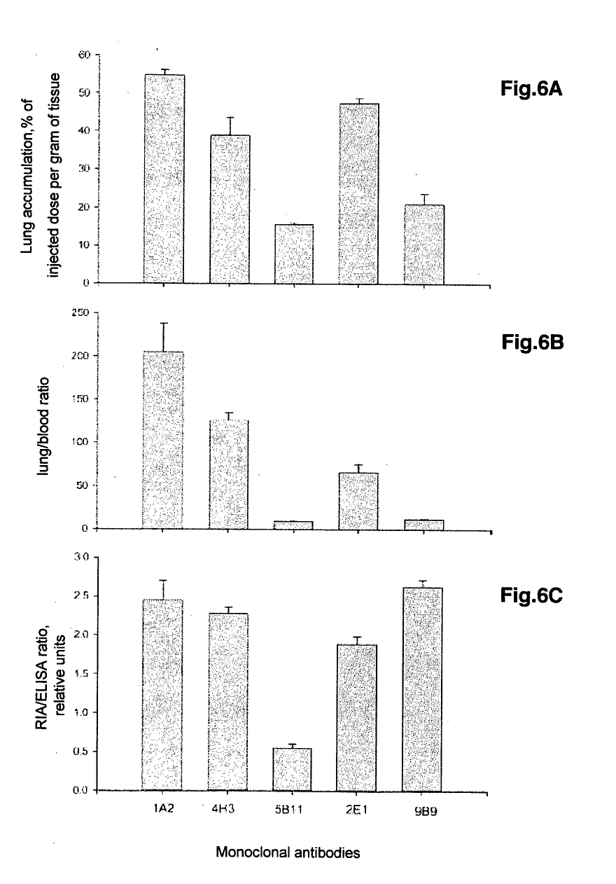

Since the ultimate proof of suitability of anti-ACE mAbs for targeting to lungs were the in vivo tests, biodistribution of radiolabeled antibodies in rats were examined. It was observed that the mAbs recognizing different epitopes of rat lung ACE accumulated selectively in the lung after systemic injection. Radiolabeled mAbs 1A2, 2E1 and 4H3 showed the highest efficacy of lung accumulation, ranging from 39 to 55% of injected dose (FIG. 6). Another important parameter characterizing the selectivity of targeting to the lungs that was also examined was the localization ratio (LR). The LR allows one to objectively compare targeting between different antibodies, since different antibodies have a different rate of uptake by target and clearing organs. It was observed that mAb 1A2 had a LR equal to 205, which was also the highest LR observed for a mAb targeting a lung antigen. The LR for mAb 4H3 and 2E1 were also relatively high, whereas the LR for mAb 5B11 did not differ from that previously obtained with 9B9 (FIG. 3B).

As described above, numerous assays were performed to characterize mAbs to rat ACE in order to understand the feature of the mAb that determined suitability for targeting to lungs in vivo, including in vitro protocols that could predict the mAb that would be most effective and selective as an affinity carrier. The results of these tests demonstrated that there was no single in vitro assay that could predict unambiguously the mAb to ACE that could be best affinity carrier for lung endothelial targeting in rats in vivo. For example, mAb 4H3 showed the highest level of binding to RLMVEC in Cell ELISA (FIG. 4A) but was the weakest in endothelial cell enrichment by magnetic beads assay (FIG. 4B). The highest efficiency in gene transfer was observed with mAb 2E1, which showed relatively low binding by ELISA (FIG. 4A). Moreover, efficacy and selectivity of radiolabeled mAb 2E1 lung uptake was higher than that for mAb 9B9 (FIG. 6), despite much lower binding to rat endothelial cells in Cell ELISA.

Hence, a combination of following parameters should determine the highest efficacy and selectivity of lung immunotargeting for each particular mAb: (1) affinity of binding, (2) rate of clearance of immunocomplexes from the blood, and (3) absence of significant effects on the shedding of immunocomplexes from the cell surface. A new mAb, Tx3.833 to lung caveolae was reported to specifically and selectively accumulate in the rat lung (McIntosh D. P. et al., 2002). The efficacy of lung accumulation of radiolabeled preparations of anti-ACE mAb 1A2 was comparable to mAb Tx3.833 that binds to a caveolae-specific antigen, 55% of ID/g versus 67% respectively. However, the selectivity of lung uptake i.e the LR for mAb Tx 3.833 was 4-fold lower than that of 1A2 (56 versus 205). Additionally, the amount of ACE in the rat lung appeared to be higher than the unknown caveolae antigen recognized by mAb Tx3.833. Half-maximal saturation of lung uptake of mAb 9B9 was achieved with 300 μg of injected mAb (Danilov et al., 1991), whereas the same effect was achieved with 7 μg of mAb Tx3.833 (McIntosh D. P. et al., 2002). Hence, mAb 1A2 could be used effectively for targeting of radiolabels to the lungs where the selectivity of tissue uptake was most crucial parameter, for example, radioimmunoimaging of lung vessels (Danilov S. et al., 1989).

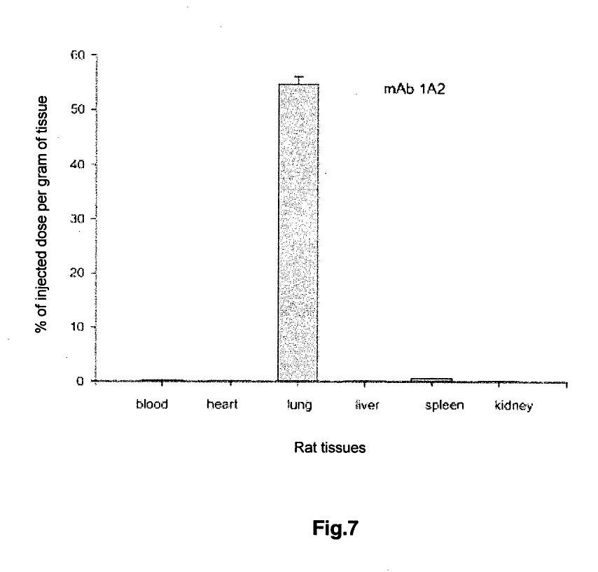

However, high selectivity of accumulation of radiolabeled mAb to rat ACE (for example for mAb 1A2) in the lung does not guarantee that this particular mAb will be most effective for drug/gene delivery. This is because selectivity of accumulation of radiolabeled mAb in the lung also depends on the tolerance of a particular mAb to radioiodination (FIG. 6C). Additionally, each of the mAbs might tolerate conjugation with drugs or vehicles used for gene delivery differently. Therefore, an accurate comparison of different native mAbs (without any chemical modifications) should be performed to consider the best mAbs for drug/gene delivery into rat lung. However, highly selective and efficient lung accumulation of radiolabeled mAb 1A2 (FIGS. 6 and 7) in combination with high rate of clearance from the blood and the high radiosensitivity made this anti-rat ACE mAb one of the best candidate not only for lung vessel imaging but also for drug/gene delivery into the rat lung endothelium.

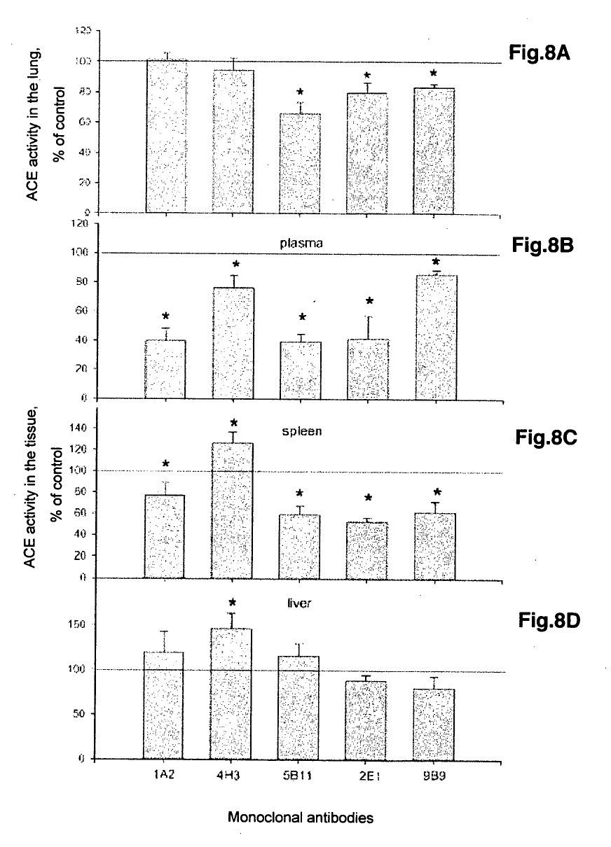

The mAbs were characterized based on their clearance from circulation when complexed with ACE. The clearance of ACE from the blood after injection of mAbs differed between mAbs (FIGS. 8B, 8C, 8D). Therefore, it is contemplated that the residual plasma ACE activity following the injection of a sufficient amount of mAb to ACE was dependent on the following parameters, a combination of which could determine ACE activity after administration of a particular antibody: (1) the proportion of the free mAb (not bound to lung endothelial ACE) and thus available to bind soluble ACE; (2) the affinity of antibody binding to soluble ACE since significant differences were observed between mAbs (FIG. 1D); (3) isotype and structure, and as a consequence the rate of clearance of the particular mAb from the circulation; (4) the efficacy of antibody-induced ACE shedding. Rat ACE complexed with mAb 4H3 was cleared from the blood more slowly than complexes of rat ACE with other mAbs since increased ACE activity was observed in the rat liver and spleen after injection of mAb 4H3. A similar pattern of antigen clearance into rat liver and spleen was observed in case of immune complexes of LPS and anti-LPS antibodies (Nys et al, 1999), antibody/DNA complexes (Edberg et al., 1989) or complexes of mAbs to tumor-associated antigens (Pimm, 1995).

In summary, the present invention generated and characterized a new set of mAbs recognizing 5 different epitopes of rat ACE. One of them, mAb1A2 demonstrated highest efficacy and selective accumulation in the lung when radiolabeled (FIGS. 6, 7) which in combination with its high rate of clearance from the blood and high radiosensitivity made this anti-rat ACE mAb one of the best candidate not only for lung vessel imaging but also for drug/gene delivery into the rat lung endothelium in different rat models of lung diseases.

The present invention is directed to a monoclonal antibody that binds specific epitope in the N-domain of ACE. Generally, the antibody is of murine origin. Most preferably, the monoclonal antibody is 1A2, 4H3, 5B11 or 2E1. Additionally, the antibody is of human origin or contains portions of a human antibody. Further, the anti-ACE monoclonal antibody may be radiolabeled.

The present invention is also directed to a method of isolating lung endothelial cells in a sample, comprising: incubating the sample with anti-ACE monoclonal antibody that binds a specific epitope in the N-domain of ACE, sedimenting the endothelial cells with magnetic beads coated with anti-mouse IgG, separating the endothelial cells that are positively labeled with the magnetic beads linked to the anti-ACE monoclonal antibody, thereby isolating the lung endothelial cells in the sample. Most preferably, the anti-ACE monoclonal antibody used in such a method is 1A2, 4H3, 5B11 or 2E1. The present invention is further directed to a method of diagnostic imaging of lung vessel, comprising: administering radiolabeled anti-ACE monoclonal antibody, where the radiolabeled anti-ACE monoclonal antibody binds a specific epitope in the N-domain of ACE on the endothelial cells in lung, detecting the bound radiolabeled anti-ACE monoclonal antibody, thereby enabling the imaging of lung vessel. Most preferably, the anti-ACE monoclonal antibody used in such a method is 1A2, 4H3, 5B11 or 2E1.

The following examples are given for the purpose of illustrating various embodiments of the invention and are not meant to limit the present invention in any fashion. One skilled in the art will appreciate that the present invention is well adapted to carry out the objects and obtain the ends mentioned, as well as those objects, ends and advantages inherent herein. Changes therein which are encompassed within the spirit of the invention as defined by the scope of the claims will occur to those skilled in the art.

### EXAMPLE 1

Purification of ACE Antigen

Rat lung ACE was purified using affinity chromatography with lisinopril-Sepharose column as described earlier (Sakharov I. Y. et al, 1991). Briefly, lung tissue was homogenized in 5 mM Tris-HCl buffer (pH 7.4) containing EDTA-free protease inhibitor cocktail and centrifuged 20,000 g for 20 min. In order to solubilize ACE from the cell membrane, pellet was re-suspended in 50mM Tris-HCl (pH 8.5) containing 1% Triton X-100 and incubated with continuous steering overnight at 4° C. After centrifugation at 20,000 g for 20 min, the supernatant was passed through 0.45uM filter and applied to lisinopril-Sepharose column. Bound ACE was eluted with 50mM Na₂B₄O₇ (pH 9.5) and dialyzed against 5mM KHPO₄ containing 10uM ZnSO₄ (pH 8.3). Human, rat and bovine ACE, purchased from Chemicon Int. (Temecula, Calif.) were used.

### EXAMPLE 2

Assay To Measure ACE Activity

ACE activity was measured as described (Piquilloud et al., 1970). Briefly, aliquots were added to 200 μl of substrate for ACE (2 mM Z-Phe-His-Leu or 5 mM Hip-His-Leu) and incubated for appropriate time at 37° C. In some experiments substrates for ACE were added directly to the wells of microtiter plates, which were coated with ACE solution similar to ELISA or plate precipitation assay. The reaction was terminated with NaOH, and the His-Leu product was estimated by incubation with O-phthaldialdehyde. The fluorescence of samples was recorded at a 365 nm excitation wavelength and a 500 nm emission wavelength.

### EXAMPLE 3

Immunization, Fusion And Screening

In order to obtain mAbs that recognized native rat ACE, mice were immunized with highly purified rat lung ACE followed by screening of hybridoma primary populations. Briefly, Balb/C female mice were immunized 6 times at one-day interval with subcutaneous injection of 7 μg rat lung ACE in complete Freund's adjuvant (CFA) following intraperitoneal (IP) injection of equal dose of the rat lung ACE in CFA next day. The titer of the antibodies in serum was tested nine days after last IP injection and the animal with the highest titer was chosen for subsequent boost. The boost was performed by intravenous (IV) injection of antigen without CFA 3 days before fusion. The fusion of the spleen cells with myeloma cell line X63.Ag8.653 was done. Hybridoma supernatants were assayed for anti-rat ACE antibodies using two assays: i) enzyme-linked immunoabsorbent assay (ELISA) on microtiter plates covered by catalytically active rat ACE in the presence of bovine serum albumin, a procedure that decreased denaturation of ACE during adsorption on plastic (Balyasnikova et al., 2003); ii) plate precipitation assay (PPA) which identifies mAbs that recognize native ACE in solution (Danilov et al., 1994). Briefly, the ELISA was performed as follows: 96-well microtiter plates (Corning, Corning, N.Y.) were coated with 50 μl rat lung ACE (5 μg/ml) in native form and incubated overnight at 4° C. After washing, 50 μl of hybridoma supernatants containing mAbs to rat ACE were added and incubated for 2 hrs at RT. The wells were then incubated with goat anti-mouse polyclonal antibodies conjugated with alkaline phosphatase (Sigma, St. Louis, Mo.) diluted 1:1000 with PBS/2% non-fat dry milk for 1 hour at RT and then washed. Alkaline phosphatase was developed using p-nitrophenyl phosphate as a substrate and read at 405 nm.

The plate precipitation assay was performed as follows: 96-well plates (Corning, Corning, N.Y.) were coated with 50 μl of affinity-purified rabbit anti-mouse IgG (10 μg/ml) overnight at 4° C. After washing with PBS/0.05% Tween 20, the wells were incubated with pure mAbs (2 μg/ml) or diluted 1/10 hybridoma culture fluids in PBS/0.1% BSA for 1 hour at RT and then washed. Wells were then incubated with 50 μl of PBS/0.1% BSA containing 20mU/ml of ACE in rat lung homogenates or serum for 2 hours at RT, washed and assayed for plate-bound ACE activity using Z-Phe-His-Leu as a substrate as described previously (Danilov S. M. et al., 1994).

From 77 primary reactive cell populations that were positive in both assays, 11 populations that showed the highest binding were chosen and cloned by limiting dilution. To exclude the clones of the hybridoma cells that were directed to identical epitopes, cross-reactivity of the mAbs to rat ACE with catalytically active ACE from sera of 15 different species were tested using a plate precipitation assay. Seven clones of hybridoma cells producing mAbs to rat ACE were further expanded for analysis. Table 1 summarizes the cross-reactivity of the chosen mAbs. MAb Ig class and subclass determination was performed using Mouse Typer® Sub-Isotyping Kit (Bio-Rad, Lab. Inc., Hercules, Calif.).

TABLE 1
Species specificity of monoclonal antibodies to rat ACE

| Species mAbs | | | | | |
|---|---|---|---|---|---|
| (Isotype) | Rat | Hamster | Cat | Rabbit | Human |
| 1A2 (IgG₁, k) | 57 | 1 | 2 | 5 | 0 |
| 2A1 (IgG₁, k) | 56 | 1 | 2 | 4 | 0 |
| 4H3 (IgG₁, k) | 90 | 10 | 1 | 4 | 0 |
| 2B11 (IgG₁, k) | 76 | 0 | 1 | 0 | 0 |
| 5B11 (IgG₁, k) | 68 | 0 | 1 | 0 | 0 |
| 2E1 (IgG_{2b}, k) | 58 | 0 | 1 | 0 | 0 |
| 2H6 (IgG₁, k) | 59 | 0 | 0 | 0 | 0 |
| 9B9 (IgG₁, k) | 100 | 22 | 87 | 1 | 344 |

Data represents the percentage of ACE activity precipitated in the well with tested mAbs. The values of non-specific absorption of ACE activity by non-immune IgG were subtracted from each value. 100% corresponds to the ACE activity precipitated from rat lung homogenate by mAb 9B9. None of the new (except mAb 9B9) studied mAbs precipitated any ACE activity from goat, sheep, dog, cow, mouse, horse, chimpanzee, macaque, swine and guinea pig.

### EXAMPLE 4

Binding of MAbs To Rat ACE

The binding pattern of anti-rat ACE mAbs to rat ACE immobilized onto plastic, to membrane-bound or soluble rat ACE in solution, or expressed on the plasma membrane of rat lung endothelial cells was compared. It was observed that the binding of most mAbs (except 2E1 and 2H6) to rat ACE absorbed on the plastic was similar to that of mAb 9B9 (FIG. 1A). The binding pattern of mAbs to rat lung ACE in solution, however, was different. The highest binding was observed with mAb 9B9 and lowest with mAb 2B11, 5B11 and 2E1 (FIG. 1B). Additionally, the binding pattern of these mAbs to soluble rat plasma ACE that lacks the transmembrane anchor was compared to membrane-bound ACE from lung homogenates. MAb 4E3 and 9B9 demonstrated the lowest degree of discrimination in binding between the two forms of rat ACE, whereas mAb 2E1 and 2H6 showed 30-50 times lower binding to soluble rat plasma ACE compared to membrane-bound ACE (FIG. 1C). It was also observed that none of the mAbs were able to bind to rat testicular ACE in the plate precipitation assay, thus indicating that the mAbs generated were directed to the N-terminal domain of rat somatic ACE. Additionally, none of the mAbs inhibited catalytic activity of rat somatic ACE in solution.

### EXAMPLE 5

Specificity of the MAb Epitope

To determine the number of different antigenic regions of rat ACE that were recognized by the panel of mAbs, the ability of different antibodies to compete with each other for binding to rat ACE was tested by performing modified plate precipitation assay as described in (Danilov S. M. et al., 1994). Briefly, the wells were coated with goat anti-mouse IgG and then with hybridoma supernatants or purified mAbs as described earlier. For the purpose of the mapping studies, the anti-rat ACE mAbs bound to the wells in this manner were designated “test antibodies”. Wells were then incubated with rat ACE (50mU/ml) that had been pre-incubated with excess amounts (50-200-fold) of another antibody, designated as “protecting antibody”. Binding of rat ACE to the test antibodies was then compared in the presence or absence of protecting antibody. In short, rat ACE was pre-incubated with excess of protecting antibody (50-200-fold) and its subsequent precipitation by test antibody was quantified. Those protecting antibodies that blocked rat ACE precipitation by the test antibody were considered to bind to the same “antigenic region” (Danilov S. M. et al., 1994). It is also known that an antigenic region (Tsartos S. J. et al., 1981) may contain numerous antigenic determinants (epitopes) separated in distance by less than the diameter of an antibody arm (35A).

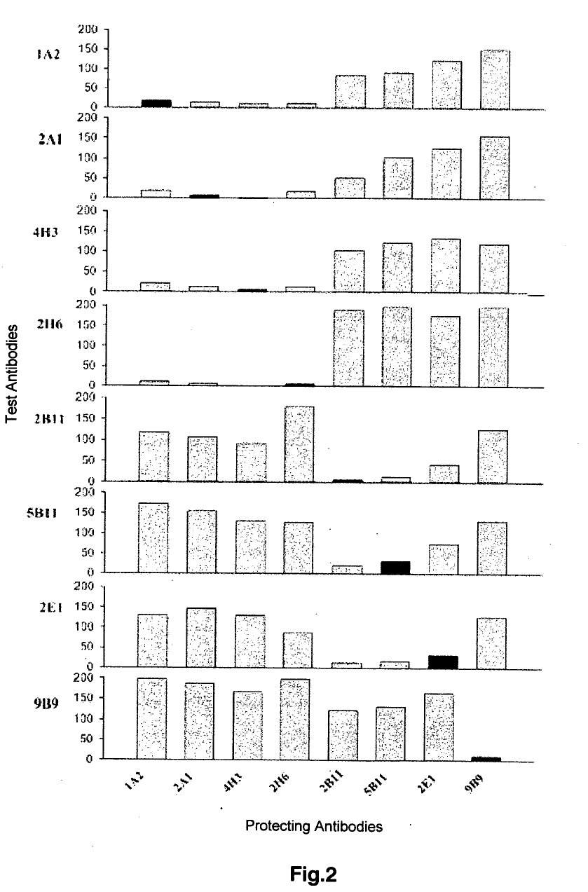

The precipitation of rat ACE by test antibody in the presence of protecting mAb is shown in FIG. 2. Typically, when the protecting mAb was the same as the test mAb or bound to an epitope overlapping that of the test antibody, >70% inhibition of ACE precipitation was observed, whereas mAbs were considered non-competitive if they demonstrated <30% inhibition (Danilov, S. M. et al., 1994; Tsartos S. J. et al., 1981). Based on these criteria, the results in FIG. 2 showed that the mAb panel revealed three antigenic regions on rat ACE: (1) region corresponding to mAb 9B9, (2) a region containing an overlapping epitopes for mAbs 1A2, 2A11, 4H3 and 2H6, (3) a region corresponding to epitopes for mAbs 2B11, 5B11 and 2E1. Within each region, complete and reciprocal inhibition was observed, consistent with the same or adjacent epitope being blocked by each mAb in the given group. However, these epitopes may not be identical since they were not equally conserved between species (Table 1) or the mAbs may behave differently in other tests.

Taken together, these results indicated that the panel of 8 mAbs recognized as many as 5 different epitopes, located in three distinct antigenic regions of the N-domain of rat ACE (FIG. 3). Therefore, only five mAbs recognizing different epitopes were further characterized.

### EXAMPLE 6

Binding of MAbs To Rat Endothelial Cells

In order to determine the mAb binding pattern with respect to the more native conformation of rat ACE, rat lung microvascular endothelial cells (RLMVEC) expressing ACE on the cell surface were used to perform Cell ELISA. Briefly, RLMVEC at passages 2^{nd} to 6^{th} growing in a gelatin-coated 96-well microtiter plates, were washed several times with PBS. Control mouse IgG or a set of anti-rat ACE mAbs in Medium 199 at concentrations 10 μg/ml were added and incubated for 1 hour at 4° C. After washing cells were fixed with 4% paraformaldehyde for 20 min at RT and washed again. Bound mAbs were quantified by incubating with goat-anti-mouse Ab conjugated with alkaline-phosphatase as described earlier. Apparent affinity constants (Kₐₚₚ) of mAbs binding to rat ACE (adsorbed to the plastic or on the surface of RLMVEC) were determined as previously described (Pinckard R. N. and Weir D. M., 1978), i.e as derived from the reciprocal of the free monoclonal antibody concentration at which 50% of the maximal binding to rat ACE was achieved.

In general, it was observed that the mAb binding patterns to cell surface rat ACE (FIG. 4A) was similar to that observed with purified rat ACE or ACE from rat lung homogenates assayed by plate precipitation assay (FIG. 1B). MAb 4H3 demonstrated the highest level of binding, whereas mAb 5B11 and 2E1 showed the lowest binding. The finding that binding of mAb 5B11 to rat lung ACE in solution (FIG. 1B) or ACE on the cell surface (FIG. 4B) was the lowest, but its binding with rat ACE adsorbed on plastic (FIG. 1A) was relatively high suggested that some degree of unmasking of some of epitope for the mAb 5B11 may have occured when ACE was adsorbed onto plastic.

Binding constants (Kₐₚₚ) for these 5 mAbs were determined using pure rat ACE adsorbed on the plastic (ELISA) or rat ACE expressed on the surface of RLMVEC (Cell ELISA). The range of Kₐₚₚ for the studied mAbs in Cell ELISA (FIG. 4A) was narrow (from 0.5×10⁸ M⁻¹ for 5B11 to 1.4×10⁸ M⁻¹ for 2E1). The relative pattern of Kₐₚₚ for tested mAbs obtained using pure rat ACE in ELISA was similar, albeit absolute values were higher.

The ability of anti-rat ACE mAbs to isolate endothelial cells from rat lungs was also examined. Briefly, the endothelial cells enrichment by magnetic beads assay was performed as follows: Lungs from sacrificed rats were cut into small slices. The lung tissue was incubated with collagenase A (1 mg/ml) at 37° C. for 2 hours. The suspension was filtered through cell dissociation sieve (Sigma, St. Louis, Mo.) composed of screens 60 and 40, and washed thrice by centrifugation at 600 g for 10 min in Hank's balanced salt solution (HBSS) Ca²⁺, Mg²⁺-free containing 0.5% BSA. The final pellet was resuspended in 1 ml of the same medium, and divided into ten equal parts. 10 μg of mAbs and control mouse IgG were added to cells chilled on ice for 30 min and incubated for another hour on the ice. Unbound antibodies were washed and cell pellet was resuspended in 100 μl of same medium containing 10⁷ M450 magnetic beads coupled with anti-mouse antibodies (Dynal Biotech ASA, Oslo, Norway). After incubating on ice for 1 hour, the endothelial cells positively labeled with magnetic beads linked to anti-rat anti-ACE mAbs were selected with the magnetic separation unit. After several washings in HBSS, fmal pellet was lysed in 100ul of 8mM CHAPS and ACE activity in lysates was estimated by enzymatic assay. Non-specific binding of beads with cells pre-incubated with control mouse IgG was subtracted. The highest recovery of ACE-expressing cells (i.e. endothelial cells) was demonstrated with mAbs 1A2 and 2E1 (FIG. 4B).

### EXAMPLE 7

Antibody-Induced Shedding of ACE From the Cell Surface

Since epitope-specific antibody-induced shedding of human ACE from the cell surface was observed previously (Balyasnikova I. V. et al. 2002, Kost O. A. et al. 2003), the ability of the new mAbs to rat ACE to induce ACE shedding from the surface of rat endothelial cell in vitro was examined. Briefly, the ACE shedding assay was performed as follows: RLMVEC growing in 96-well microtiter plates were washed thrice with HBSS and incubated with mAbs (10 μg/ml) diluted in serum-free culture medium. The culture medium was collected after 6 hours. In order to determine the rate of ACE release, supernatants were centrifuged and ACE activity was measured using fluorimetric assay. Aliquots (50 μl) were added to 200 μl of 5mM Hip-His-Leu (substrate for ACE) and incubated for appropriate time at 37° C. The reaction was terminated with NaOH, and the His-Leu product was estimated fluorimetrically (Friedland J. and Silverstein E. A., 1976).

MAbs 1A2 and 4H3 significantly inhibited ACE release from the cell surface by 29% and 26% respectively (FIG. 4C). MAbs 5B11 and 2E1 had no effect on ACE shedding, whereas mAb 2H6 induced ACE shedding to a similar degree as mAb 9B9. Since none of the assayed mAb inhibited enzymatic activity of soluble ACE, a decrease in ACE activity in the culture fluid after incubation of endothelial cells with mAb 1A2 or 4E3 could be due to inhibition of ACE shedding rather than an anti-catalytic effect of these mAbs.

### EXAMPLE 8

In Vitro Gene Delivery Via ACE

A dramatic increase in the efficacy and specificity of gene delivery in vitro (CHO-ACE cells) and in vivo (into rat lung endothelium) using adenoviruses coated with anti-ACE mAb 9B9 was demonstrated previously (Reynolds P. N. et al., 2000; Reynolds P. N. et al., 2001). Therefore, the ability of these mAbs to enhance gene delivery in vivo was examined in order to select the most effective mAb for rat lung gene delivery in vivo. Suitability of mAb 9B9 for gene delivery into rat lung was demonstrated by a preparation of bi-specific conjugate of 9B9 with a mAb to adenoviral surface protein. However, since this approach was very labor intensive and impractical for comparing several antibodies, the mAbs of the present invention were examined based on their ability to increase transduction of rat microvascular endothelial cells in culture. A new type of adenovirus that carried a C-binding domain of protein A on its surface (Khorokhov et al., 2003) and allowed immunoglobulins to attach to the viral surface without chemical modification of mAbs was used (Douglas et al., 1996). Briefly, the in vitro gene delivery was performed as follows: RLMVEC from passage 2 to 6 were plated in 24-well plates at a density of 100,000 cells per well. The cells were infected 24 hour later with adenoviral vector Ad5DRLLCd, containing Ig-binding C domain of protein A (Khorokhov N. et al., 2003). Adenoviral vector infection of the cells was performed using a multiplicity of infection (MOI) of 250 plaque forming units (pfu/cell) diluted from stock in HEPES-buffered saline (HBS; 150mmol/L HEPES, 20mmol/L NaCl, pH 7.8). In order to form antibody-virus complexes, Ad5DRLLCd were mixed with different mAbs to rat ACE or diluent control and incubated at room temperature for 15 minutes in a total volume of 100 μl. After initial optimization studies in which a fixed dose of virus was complexed to serially diluted amounts of mAbs, an optimal ratio of 2.5×10⁹ pfu Ad: 3-30ng mAbs was determined. Prior to infection, the volume of each mixture was brought to 200 μl with warmed (37° C.) DMEM/F12 medium and added to the cells (200 μl per well in triplicate). The infected media were then aspirated, followed by washing of the cells once with Dulbecco's phosphate buffered saline (D-PBS, Cellgro) and addition of 1 ml of appropriate complete media with 10% FBS to each well. Cells were further incubated for 48 hours and luciferase reporter gene expression was assayed using Luciferase Assay System kit (Promega, Madison, Wis.). mAbs to rat ACE increased (30-50folds) the transduction of rat endothelial cells by adenoviruses (FIG. 4D). The transduction was epitope-dependent and the mAbs were effective in a very narrow concentration range. However, binding of mAbs to the surface of this particular adenovirus (with C-domain of protein A) was inhibited by high concentration of immunoglobulins present in rat blood. Therefore, this protocol has to be refmed to confirm the ability of these mAbs to rat ACE to enhance gene delivery into rat endothelial cells in vivo.

### EXAMPLE 9

Immunohistochemistry of ACE In Rat Lung

The mAbs were characterized based on their ability to detect rat lung ACE by immunostaining of ACE by the alkaline phosphatase anti-alkaline phosphatase (APAAP) technique as described (Metzger et al., 1999). The following were the aims of this characterization: (1) compare the binding of different mAbs in the lung under identical conditions, (2) evaluate mAb binding sites in other organs, (3) determine the cell types recognized by the different mAbs, and (4) reveal potential heterogeneity of mAb binding to endothelial cell in the microcirculation (Danilov et al., 1994). Untreated, fresh frozen tissues were used to ensure maximum preservation of the epitope. Briefly, frozen tissue sections were air dried and fixed in acetone for 10 min at room temperature (RT). Sections were incubated with the primary mAb followed by the secondary rabbit anti-mouse immunoglobulin (1:40, Dako) supplemented with reconstituted lyophilized rat serum (1:700, Dianova) and the APAAP complex (1:50, Dako) where each step lasted for 30 minutes at RT. Alkaline phosphatase substrate reaction with fuchsin (100 μg/ml) and levamisole (400 μg/ml) was performed for 20 min at RT. Sections were counterstained with hematoxylin and mounted in gelatin. All mAbs were tested on various native rat tissues to determine the optimal concentration (Table 2). Immunoreactivities were compared to those of different monoclonal markers raised against endothelial cells (clones HIS52, TLD-3A12) and histiocytes (clones ED1, ED2). A non-specific mAb (clone MR12/53) served as negative control.

TABLE 2
Antibodies to rat ACE and controls
used in immunohistochemistry

| | | Concentration^{a} | |
|---|---|---|---|
| Clone | Immunogen | (μg/ml) | Source |
| 1A2 | Rat ACE | 1.0 | This study |
| 2A1 | Rat ACE | 0.5 | * |
| 2B11 | Rat ACE | 2.0 | * |
| 5B11 | Rat ACE | 5.0 | * |
| 2E1 | Rat ACE | 5.0 | * |
| 2H6 | Rat ACE | 50.0 | * |
| 4H3 | Rat ACE | 1.0 | * |
| 9B9 | Human ACE | 1.0 | Danilov et al (1994) |
| ED1 | CD68 | 0.05 | Serotec, Ltd |
| ED2 | CD163 | 0.05 | * |
| HIS52 | Endothelium | 1:5000 | Serotec Ltd |
| | (RECA-1 antigen) | | |
| TLD-3A12 | Endothelium | 0.1 | BD Pharmingen |
| | (CD31, PECAM-1) | | |
| MR12/53^{b} | Rabbit IgG | 1.0 | DAKO |

^{a}Concentrations of mAbs used for immunostaining, shown in FIG. 5, are first saturating concentrations determined by the tittration of the immunostaining.
^{b}Used as negative control in APAAP-technique

The entire set of mAbs to rat ACE gave a uniform reaction pattern in the rat lung (FIGS. 5A-5D). Furthermore, all of them showed an identical staining pattern to that of mAb 9B9 (FIG. 5E). In the lung, endothelial cells showed the highest degree of mAb binding and to a lesser extent, ACE was also detected in fibroblasts and in activated alveolar macrophages. With minor exceptions, mAbs labeled almost the entire vasculature endothelium in rat lung, including pulmonary arteries, pulmonary veins and the microcirculation. In contrast to the endothelial marker CD31, the following minor labeling differences were observed: pulmonary veins and the alveolar microcirculation were labeled strongly by mAbs to ACE than pulmonary arteries.

The endothelial cells of bronchial vasculature showed an ACE expression pattern characteristic of systemic circulation, where only 10% of capillary endothelial cells were ACE-positive (Franke F. E. et al., 1997), compared to the alveolar microvessels, where 100% of endothelial cells expressed ACE. Since capillaries represented the most extended vascular surface in any organ, the binding ability of these anti-ACE mAbs in capillaries of different organs were characterized. In contrast to other organs and compared to pan-endothelial controls, the rat lung displayed an almost robust and uniform ACE expression in the endothelium of the entire alveolar microcirculation. These results indicated that that these mAbs to rat ACE might be suitable for rat lung targeting in vivo as was demonstrated previously with mAb 9B9.

### EXAMPLE 10

Targeting To Lungs By MAbs To Rat ACE

Since exposure of tissue sections to antibodies for a sufficient time allows for binding to maximal number of intra- and extracellular ACE epitopes, immunohistochemical studies do not provide insights into the antigens ability to harbor ligands circulating in the bloodstream. Therefore, in order to address this issue directly and to estimate the accessibility of different epitopes of ACE to circulating antibodies, the uptake of ¹²⁵I-mAbs after systemic injection was measured.

Radioiodination of antibodies with ¹²⁵I was performed in Iodo-Gen precoated tubes (Pierce, Rockford, USA) as described by the manufacturer. Briefly, 100 μg of antibody was incubated for 10 min on ice with 100 μCi of Na¹²⁵I. Excess iodine was removed by gel filtration on a PD-10 (Sephadex G-25) mini-column (Pharmacia, Uppsala, Sweden).

Male Sprague-Dawley rats were injected with antibodies (0.5 μCi) via the tail vein under halothane anesthesia. After 2 hours, the animals were sacrificed and the internal organs were removed, washed, blotted and weighed. Tissue radioactivity was determined gamma scintillation counter. Results were expressed as follows: 1) % injected dose (ID)—radioactivity in organ versus radioactivity in the injected dose; 2) % ID per gram of tissue (reflects the efficiency of antibody uptake); 3) Localization ratio (LR)—the ratio of radioactivity in gram of tissue to ml of blood. The LR shows tissue selectivity of antibody uptake (Danilov S. et al., 1989).

All ACE mAbs accumulated specifically in the lung after systemic injection (FIG. 6A). MAb 1A2, 2E1 and 4H3 demonstrated the highest efficacy of lung accumulation (55, 48 and 39% of injected dose per gram of tissue, respectively). The efficiency of lung accumulation of mAb 5B11 was comparable to mAb 9b9 (around 20% of ID/g). MAb 1A2 showed the highest selectivity of lung accumulation expressed as LR (i.e. the ratio of organ radioactivity to blood radioactivity) equal to 205. The LR for mAb 2E1 and 4H3 was in the range of 66-130 and mAb 5B11 was similar to that of 9B9 i.e. around 10 (FIG. 6B).

Furthermore, the resistance of antibodies to iodination was investigated and expressed as a ratio of the binding of native mAbs to rat ACE to that of radiolabeled mAbs. Briefly, to determine binding of ¹²⁵I-labeled mAbs to ACE immobilized on plastic, 96-well microtiter plates were covered with 1:20 dilution of rat lung ACE (50 μl, 5 μg/ml) overnight at 4° C. and washed with PBS/Tween 20. ¹²⁵I-labeled control mouse IgG or anti-ACE mAbs in PBS/0.1% BSA was added to immobilize ACE and the plates were further incubated for 1 hour at RT. After washing, the ¹²⁵I-mAb bound to ACE immobilized on the plastic was eluted by hot 10% SDS and measured with gamma-counter. MAbs 1A2, 4H3 and 9B9 were the most resistant to iodination (FIG. 6C), while mAb 5B11 showed the highest sensitivity to iodination. Further, the accumulation of mAb 1A2 in other tissues after systemic injection was negligent compared to the remarkably efficient and selective uptake in the lung (FIG. 7).

### EXAMPLE 11

The Effect of Injection of MAbs On Rat ACE

In order to estimate the effect of systemic injection of mAbs on ACE activity in pulmonary circulation, i.e., primarily due to ACE shedding (Danilov S. et al., 1994), all antibodies along with control mouse IgG were injected intravenously at a concentration of 0.5 or 3 mg/rat. Although none of the assayed mAbs showed a direct inhibitory effect on enzymatic ACE activity, mAbs 2E1, 9B9 and 5B11 significantly decreased ACE activity in the lung by 20-30% compared to the control. The other mAbs that were examined had no effect on the level of ACE in the lung (FIG. 8A).

The level of ACE in plasma after mAb injection was also examined to address the issue of ACE clearance as an antigen-antibody complex from the rat circulation. MAbs 4H3 and 9B9 showed the lowest ACE clearance from plasma-15-25% below control, whereas injection of mAb 1A2, 5B11 and 2E1 induced a dramatic increase in ACE clearance from the circulation-30-45% of ACE activity in the plasma compared to control animals (FIG. 8B). Injection of these mAbs decreased ACE activity in the spleen (FIG. 8C), whereas ACE activity increased by 25% in the spleen and 40% in the liver of animals injected with mAb 4H3 (FIG. 8D).

The following references were cited herein:
Atochina et al., (1998) pi Am J Physiol Lung Cell Mol Physiol 275, L806-L817.
Balyasnikova, I. V. et al (1998) In Vitro Cell. Dev. Biol. 34, 545-554.
Balyasnikova, I. V. et al. (1999) Tumor Targeting 4, 70-83.
Balyasnikova, I. V. et al. (2002) Biochem J 362, 585-595.
Balyasnikova, I. V. et al. (2003) Tissue Antigens 61, 49-62.
Battle, T. et al. (1994) Tissue Cell 26, 943-955.
Brosnan M. J. et al. (2004) in press
Caldwell, P. R. et al. (1976) Science 191, 1050-1051.
Chesterman, C. N. et al. (1983) J Cell Physiol 116, 45-50.
Corvol, P. et al. (1995) Meth Enzymol 248, 283-305.
Danilov, S. et al. (1994) Int Immunol 6, 1153-1160.
Danilov, S. et al. (1989) J Nucl Med 30, 1686-1692.
Danilov et al. (2001) Am J Physiol Lung Cell Mol Physiol 280, L1335-L1347.
Danilov, S. M. et al. (1994) J Biol Chem 269, 26806-26814.
Danilov, S. M. et al. (1991) Lab Invest 64, 118-124.
Danilov, S. M. et al. (1988) Doklady Acad Sci USSR (Russian) 301, 1003-1007.
Del Vecchio and Smith (1982) Cell Biol Int Rep 6, 379-384.
Douglas, J. T. et al. (1996) Nature Biotech 14, 1574-1578.
Edberg, J. C. et al. (1989) Clin Immunol Immunopathol 51: 118-32.
Ehlers, M and Riordan, J. (1989) Biochemistry 28, 5311-5318.
Franke et al. (1997) Angiotensin I-converting enzyme (CD 143) on endothelial cells in normal and in pathological conditions. In: Kishimoto et al. eds. Leucocyte Typing VI: White Cell Differentiation Antigens. Garland Publishing Inc. New York, 1997: 749-51.
Friedland and Silverstein (1976) Am J Clin Path 66, 416-424.
Johnson, A. R. et al. (1980) Clin Exper Hypertension 2, 659-674.
Khorokov, N. et al. (2003) J Virol 77, 12931-12940.
Kohler, G and Milstein, C. (1975) Nature 256, 495-497.
Kost, O.A. et al. (2003) Biochemistry 42, 6965-6976.
McIntosh, D. P. et al. (2002) Proc Natl Acad Sci USA 99, 1996-2001.
Metzger, R. et al. (1999) Kidney Int 56, 1442-1454.
Morrel, N. W. et al. (1995) J Clin Invest 96, 1823-33.
Muzykantov, V. et al. (1994) J Nucl Med 35, 1358-1365.
Muzykantov et al. (1996) Am J Physiol Lung Cell Mol Physiol 270, L704-L713.
Muzykantov, V. et al. (1996) J Pharm Exp Therap 279, 1026-1034.
Muzykantov, V. R. and Danilov, S. M. (1995) Targeting of radiolabeled monoclonal antibody against angiotensin-converting enzyme to the pulmonary vasculature. In: Torchilin V P (ed). Handbook of Targeting Delivery of Imaging Agents. CRC, Boca Raton, Fla., pp 465-486.
Muzykantov, V. R. et al. (1996) Proc Natl Acad Sci USA 93, 5213-5218.
Nys, M. et al. (1999) Med Microbiol Immunol 188, 65-71.
Ohno, K. et al. (1997) Nature Biotech 15, 763-767.
Piquilloud, Y. et al. (1970) Biochim. Biophys Acta 206, 136-142.
Pimm, M. V. (1995) Nucl Med Biol 22, 137-145.
Pinckard, R. N. and Weir, D. M. (1978) Chapter 16, In: Weir, D. M. ed. Handbook of Experimental Immunology. Blackwell Scientific, Oxford, UK 16.1-16.20.
Reynolds, P. N. et al. (2001) Nature Biotech 19, 838-842.
Reynolds, P. N. et al. (2000) Mol Ther 2, 562-578.
Ried, M. U. et al. (2002) J Virol 76, 4559-4566.
Rosen, E. M. et al. (1985) J Cell Physiol 122, 30-38.
Ryan, U. S. et al. (1976) Tissue Cell 8, 125-145.
Sakharov, I. Y. et al. (1991) Biochemistry (Moscow) 56, 55-62.
Soffer, R. L. (1976) Ann Rev Biochem 45, 73-94.
Tsartos, S. J. et al. (1981) J Biol Chem 256, 8635-8645.
Volpers, C. et al. (2003) J Virol 77, 2093-2104.
Wickham, T. J. et al. (1996) J Virol 70, 6831-6838.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

1. A monoclonal antibody that binds a specific epitope in the N-domain of ACE.

2. The monoclonal antibody of claim 1, wherein said antibody is of murine origin.

3. The monoclonal antibody of claim 2, wherein said murine monoclonal antibody is 1A2, 4H3, 5B11 or 2E1.

4. The monoclonal antibody of claim 1, wherein said antibody is of human origin or contains portions of a human antibody.

5. The monoclonal antibody of claim 1, wherein said antibody is radiolabeled.

6. A method of isolating lung endothelial cells in a sample, comprising:
incubating said sample with anti-ACE monoclonal antibody that binds a specific epitope in the N-domain of ACE;
sedimenting the endothelial cells with magnetic beads coated with anti-mouse IgG;
separating the endothelial cells that are positively labeled with said magnetic beads linked to said anti-ACE monoclonal antibody, thereby isolating said lung endothelial cells in said sample.

7. The method of claim 6, wherein said anti-ACE monoclonal antibody is 1A2, 4H3, 5B11 or 2E1.

8. A method of diagnostic imaging of lung tissue, comprising:
administering radiolabeled anti-ACE monoclonal antibody, wherein said radiolabeled anti-ACE monoclonal antibody binds a specific epitope in the N-domain of ACE on the endothelial cells in lung;
detecting said bound radiolabeled anti-ACE monoclonal antibody, so as to image said lung tissue.

9. The method of claim 8, wherein said anti-ACE monoclonal antibody is 1A2, 4H3, 5B11 or 2E1.
